(19) United States
(12) Patent Application Publication
(10) Pub. No.: US 2025/0121122 A1
(43) Pub. Date: Apr. 17, 2025

(54) Blood Treatment Apparatus with Vibration Device

(71) Applicant: Fresenius Medical Care Deutschland GmbH, Bad Homburg (DE)

(72) Inventors: Martin Urban, Bad Homburg (DE); Thomas Nuernberger, Bad Homburg (DE); Leonardo Fiume, Bad Homburg (DE)

(21) Appl. No.: 18/834,087

(22) PCT Filed: Jan. 30, 2023
(86) PCT No.: PCT/EP2023/052149

§ 371 Date: Jul. 29, 2024

(30) Foreign Application Priority Data
Jan. 31, 2022 (DE) 10 2022 102 164.5

(51) Int. Cl. A61M 1/16; A61M 1/14; A61M 1/36

(52) U.S. Cl. CPC A61M 1/1601; A61M 1/15; A61M 1/3622; A61M 1/3626; A61M 1/363; A61M 2202/0021; A61M 2202/0413; A61M 2205/10; A61M 2205/12; A61M 2205/18; A61M 2205/3334; A61M 2205/3344; A61M 2205/3379; A61M 2205/50; A61M 2205/75

(57) ABSTRACT

The present disclosure relates to a blood treatment apparatus having or connected to at least one blood pump, an air bubble detector, another pump, a vibration device and a control device or closed-loop control device. The blood pump is provided for pumping blood through an extracorporeal blood circuit during a blood treatment session, during which the blood treatment apparatus is connected to the extracorporeal blood circuit and to a blood filter. The air bubble detector is provided for detecting air bubbles within the extracorporeal blood circuit. The additional pump is provided for conveying dialysis liquid and/or dialysate. The vibration device is provided and/or is suitable for causing the extracorporeal blood circuit, a section thereof, or its contents, to vibrate. The control device or closed-loop control device is provided and programmed to control the blood pump, the additional pump for conveying dialysis liquid and/or dialysate and the vibration device.

### CROSS-REFERENCE TO RELATED APPLICATIONS

The present application is the national stage entry of International Patent Application No. PCT/EP2023/052149, filed on Jan. 30, 2023, and claims priority to Application No. DE102022102164.5, filed in the Federal Republic of Germany on Jan. 31, 2022, the disclosures of which are expressly incorporated herein in their entirety by reference thereto.

### TECHNICAL FIELD The present disclosure relates to a blood treatment apparatus.

### BACKGROUND

Varying forms of blood treatment apparatuses are known from practice. They include, for example, devices for hemodialysis, hemofiltration and hemodiafiltration. During extracorporeal blood treatment, blood flows in an extracorporeal blood circuit through a blood treatment unit. In the devices for hemodialysis, hemofiltration and hemodiafiltration, the blood treatment unit is a dialyzer or filter which, in simple terms, is separated by a semi-permeable membrane into a blood chamber and a dialysis liquid chamber. During blood treatment by hemodialysis or hemodiafiltration, the blood flows through the blood chamber while a dialysis liquid flows through the dialysis liquid chamber.

Air bubbles or micro-bubbles in extracorporeal circuits downstream of a venous air separation chamber can lead to gas embolisms in the patient's body. Emboli can clog vessels and cause ischaemia. To prevent gas from entering the patient's vascular system during dialysis, the venous air separation chamber is located in the venous blood tubing system. It partially reduces the flow velocity within the extracorporeal circuit so that gas bubbles can rise against gravity according to Archimedes' principle and may be separated through an opening in an upper area of the venous air separation chamber.

In addition, the venous system downstream of the venous air separation chamber is monitored by an air bubble detector for the presence of air, e.g. in the form of micro-bubbles, in the extracorporeally flowing blood. If the sensor detects the presence of air, an air bubble alarm is issued, which interrupts the patient's blood treatment. Once the cause of the air bubble alarm has been remedied by the person in charge, the blood treatment can be continued.

### SUMMARY

In an aspect, the present disclosure proposes a further blood treatment apparatus with an air bubble detector.

According to the present disclosure, a blood treatment includes or is connected to a respective blood pump and air bubble detector for an extracorporeal blood circuit, and a pump for conveying dialysis liquid and/or dialysate on its hydraulic side. The extracorporeal blood circuit, which is not part of the blood treatment apparatus but is set up or connected to it for the purpose of blood treatment of a specific patient before the start of his blood treatment session, may be provided wholly or partly on a blood cassette.

The blood pump is provided for pumping blood through the extracorporeal blood circuit or through the blood cassette during a blood treatment session when connected to the extracorporeal blood circuit and to a blood filter or dialyzer, the dialyzer in turn having a semi-permeable membrane.

The air bubble detector serves to detect air bubbles within the extracorporeal blood circuit. The air bubble detector can be arranged, for example, between a venous air separation chamber and the venous patient access.

The blood treatment apparatus further includes a vibration device by which the extracorporeal blood circuit or at least a section thereof, or respectively the contents or a part of the contents of the blood circuit or section, can be set in motion, vibrated, moved or oscillated.

The blood treatment apparatus further includes or is connected to a control device or closed-loop control device. The control device or closed-loop control device is configured to initiate, perform, control and/or regulate a method, e.g., automatically, in interaction with the blood treatment apparatus or its equipment or devices, e.g., as disclosed herein. For example, the control device or closed-loop control device is configured to control the blood pump, the pump for dialysis liquid and/or dialysate, and to control the vibration device.

An interaction may be or include actuation, control or regulation. An interaction may be or require a signal connection.

For the method disclosed herein, the blood treatment apparatus according to the present disclosure may include or be connected to a correspondingly suitable and/or configured device or apparatus, such as a pressure measuring device for measuring pressure or the like.

In all of the statements herein, the use of the expression “may be” and “may have” etc. is synonymous to “is preferably” or “has preferably,” etc. respectively, and is intended to illustrate an embodiment according to the present disclosure.

Whenever numerical words are mentioned herein, the person skilled in the art shall recognize or understand them as indications of a numerical lower limit. Unless it leads the person skilled in the art to an evident contradiction, the person skilled in the art shall comprehend the specification for example of “one” (also “a/an”) as encompassing “at least one”. This understanding is also equally encompassed by the present disclosure as the interpretation that a numeric word, for example, “one” (also “a/an”) may alternatively mean “exactly one”, wherever this is evidently technically possible for the person skilled in the art. Both understandings are encompassed by the present disclosure and apply herein to all used numerical words.

When “programmed” or “configured” is mentioned herein, it is also disclosed that these terms are interchangeable.

When referring herein to a venous air separation chamber, or also briefly to an air separation chamber, this can also be a venous blood chamber, a venous bubble chamber or a venous drip chamber.

Whenever an embodiment is mentioned herein, it is then an exemplary embodiment.

When it is disclosed herein that the subject-matter according to the present disclosure includes one or several features in a certain embodiment, it is also respectively disclosed herein that the subject-matter according to the present disclosure does, in other embodiments, likewise according to the present disclosure, explicitly not include this or these features, for example, in the sense of a disclaimer. Therefore, for every embodiment mentioned herein it applies that the converse embodiment, e.g. formulated as negation, is also disclosed.

Embodiments according to the present disclosure may include some or more of the features mentioned above and/or following in each possible technical combination.

In some embodiments, the vibration device of the blood treatment apparatus according to the present disclosure is arranged in or on the extracorporeal blood circuit, or with effect thereon, with the aim of vibrating the extracorporeal blood circuit, at least a section thereof or, respectively, the contents thereof, which is mostly caused by the correspondingly programmed control device or closed-loop control device.

In some embodiments, the vibration device is not (or not only) the venous tubing clamp, at least not unless the control device or closed-loop control device is correspondingly programmed to cause the venous tubing clamp to vibrate by a sufficiently rapid opening and closing, e.g. as described herein.

In some embodiments, the vibration device is or includes a venous tubing clamp. Hereby, the control device or closed-loop control device is programmed to actuate the venous tubing clamp such that it is automatically opened and closed or otherwise actuated multiple times within a minute or second. This action of the venous tubing clamp can occur at a rate of at least once per second. When opening and/or closing the venous tubing clamp, vibrations can be transmitted to the tubing system via this.

In certain embodiments, in which the vibration device is a venous tubing clamp, vibrations of the tubing system can be initiated by the pressures or negative pressures generated during opening and closing of the tubing clamp.

In some embodiments, the vibration device is or includes a holding device for releasably holding at least a portion of the extracorporeal blood circuit, e.g., to a housing surface of the blood treatment apparatus. The holding device may be a clamp for holding a flexible tubing section, the drip chamber, or another section of the disposable.

In some embodiments, the vibration device is configured and controlled accordingly to vibrate at a frequency of 1 Hz, 5 Hz or more.

In some embodiments a vibration is a left-right movement or an up-down movement. In some implementations, the vibration is not about a longitudinal or transverse axis and/or does not represent a rotational movement.

In some embodiments of the blood treatment apparatus according to the present disclosure, the control device or closed-loop control device is configured to modulate the frequency of the vibrations generated by the vibration device. This can be done, for example, by a frequency sweep, amplitude modulation and/or resonance excitation or can include such methods and can advantageously contribute to removing the air within the extracorporeal blood circuit as effectively as possible. Hereby, the resonance behavior of the air and/or the liquid is used. The direction of the vibration is not limited here and can be both radial and linear, for example, in the direction of the venous air separation chamber, or in any spatial direction, from which certain directions (e.g., buoyancy direction) may be used.

In some embodiments, the control device or closed-loop control device is programmed or configured to carry out the steps of one of the two alternative embodiments of the following method during an extracorporeal blood treatment session after detecting air bubbles or air pockets in the extracorporeal blood circuit, or after an air bubble alarm.

In example embodiments, the method includes stopping the blood pump or reducing its conveying rate, with which it conveyed immediately before the detection of the air bubbles, and generating a negative transmembrane pressure across the semi-permeable membrane of the blood filter. This negative transmembrane pressure can be achieved by appropriately controlling the pump for conveying dialysis liquid and/or dialysate on the hydraulic side while the blood pump is stopped or its conveying rate is reduced.

The transmembrane pressure can be measured in a variety of ways or, respectively, its existence can be determined directly or indirectly.

Air bubbles or air pockets in the extracorporeal blood circuit can be detected using the air bubble detector. Alternatively or additionally, the air bubble detector can be suitable or configured to trigger an air bubble alarm (alternatively: gas alarm) as a result of the detection of gas, air or air bubbles, which in turn causes the method to be initiated or carried out.

Air bubbles or air pockets can be detected, for example, by an air bubble alarm, by the fulfillment of conditions that trigger an air bubble alarm or are sufficient to trigger an air bubble alarm.

In some embodiments, the method encompasses that the conveying direction of the blood pump is reversed, that is to say it conveys backwards.

In the method carried out using the control device or closed-loop control device of the blood treatment apparatus according to the present disclosure, a negative transmembrane pressure is generated across the semi-permeable membrane in some embodiments with the venous tubing clamp fully or partially closed.

In some embodiments, the method encompasses, as a further step, opening the venous tubing clamp when a negative transmembrane pressure has already been generated or is present, while maintaining a negative transmembrane pressure.

Due to the negative transmembrane pressure, a (transmembrane) plasma water transfer takes place via the semi-permeable membrane and the blood in the dialyzer is hemoconcentrated. Hereby, the volume of liquid that is pumped into the hydraulic system or that is shifted from the extracorporeal blood circuit to the hydraulic side via the semi-permeable membrane due to the pressure difference is replaced by blood, which due to the hemoconcentration and negative pressure is made to flow in the opposite (herein also: retrograde) direction to its usual flow direction within the blood circuit via the venous part of the tubing system from the latter and from the patient's vascular system. Thereby, the air bubbles or air pockets (e.g., gas bubbles and micro-bubbles) present in the line are retrograde transported back into the venous air separation chamber and separated there, mostly to the environment.

In some embodiments, the venous tubing clamp is opened if a minimum value of a negative transmembrane pressure has been detected by a pressure sensor of the blood treatment apparatus.

In some embodiments, a negative transmembrane pressure is generated by the pump on the hydraulic side of the blood treatment apparatus while an optional substitute fluid pump of the blood treatment apparatus is stopped or not conveying.

In some embodiments, generating or maintaining the negative transmembrane pressure is terminated by the control device or closed-loop control device as soon as a negative transmembrane pressure of about −300 mmHg to −500 mmHg is reached, or when a volume of at least 20 ml to 60 ml of blood has been conveyed past the air bubble detector towards the venous air separation chamber by the generated transmembrane pressure.

In some embodiments, conveying by the pump on the hydraulic side of the blood treatment apparatus to generate a negative transmembrane pressure is terminated after the air bubble detector no longer detects air bubbles or air pockets and/or there is no longer an air bubble alarm and/or at least 20 ml to 60 ml of blood has been retrograde conveyed.

In some embodiments of the blood treatment apparatus, the control device or closed-loop control device is programmed to initiate some or all of the method steps disclosed herein in any combination, at least overlapping in time.

In some embodiments, the pump for conveying dialysis liquid and/or dialysate is an optionally provided ultrafiltration pump by which, for example, a negative transmembrane pressure can be generated, which in turn can bring about reverse conveying of blood in the extracorporeal blood circuit.

In some embodiments, the blood treatment apparatus according to the present disclosure is connected to an extracorporeal blood circuit and to a blood filter or dialyzer with a semi-permeable membrane.

In some embodiments, the extracorporeal blood circuit is a blood tubing set and/or a blood cassette or includes a blood tubing set and/or a blood cassette.

In some embodiments, the blood treatment apparatus is configured as a hemodialysis device, a hemofiltration device, a hemodiafiltration device or a device for performing a separation procedure.

In some embodiments, the method initiated by the control device or closed-loop control device encompasses determining whether the negative transmembrane pressure generated or its amount is within predetermined limits, exceeds or falls below a limit value, exceeds a minimum value and/or does not exceed a maximum value. This can be done on the basis of at least one criterion (limit value, range, maximum value, etc.), which can be stored, for example, in a memory device, e.g. of the blood treatment apparatus.

In some embodiments, the method initiated by the control device or closed-loop control device encompasses emitting or outputting an audible and/or optical or otherwise signal-connected air bubble alarm only if, during or after the method has been completed, there is a new condition or still is a condition which would have already led to an air bubble alarm for the person in charge and/or the blood treatment apparatus before the start of the method. It can thus be provided that the user is only informed optically and/or audibly about the presence of an air bubble alarm and/or that the blood treatment is only automatically interrupted if, after detection of air bubbles, the removal of the detected air via the method, which is carried according to the present disclosure out by the blood treatment apparatus, was not successful.

In some embodiments, the further pump is a positive displacement pump, e.g., a diaphragm pump, eccentric diaphragm pump, peristaltic pump, roller pump or piston pump.

Where reference is made herein to a signal connection or communication connection between two components, this may be understood to mean a connection that exists in use.

In some embodiments, the method disclosed herein that is initiated by the control device or closed-loop control device may encompass, as a further optional step, causing an oscillation, movement, vibration or the like. This step can be carried out simultaneously or overlapping with other parts of the method as described herein. Causing a vibration, movement, oscillation or the like may be in all embodiments of the disclosed method.

In some embodiments, the blood treatment apparatus does not include a drive device that is arranged and/or controlled to cause a rotation, a shaking motion or a vibration of the dialyzer or blood filter. In some implementations, the control device or closed-loop control device may not be configured to cause such rotation (e.g. about a longitudinal axis or about a transverse axis), shaking motion or vibration.

By using embodiments according to the present disclosure, one or more of the advantages mentioned herein may be achievable, which include the following:

Via the solution according to the present disclosure, a procedure with considerable effort on the part of the user in the event of an air bubble alarm can be advantageously avoided. In the conventional case, the user would have to disconnect the patient venously, which is associated with a hygienically critical loosening of a tubing connection of at least one tubing segment filled with whole blood, and then connecting the venous tubing system, e.g., with a saline bag, in order to pump the blood volume of the venous line, in which the detected air bubbles or air pockets are present, into the saline bag at a low flow rate. With this conventional method, when the user realises that the line is now gas-free, he reconnects the venous line to the patient and continues the treatment. Thus, using the systems, methods, and devices of the present disclosure, both the treatment can be simplified and patient safety can be further increased.

Another advantage of the present disclosure may be that the patient does not suffer any blood loss by discarding whole blood, e.g., into the aforementioned saline bag, during the procedure according to the present disclosure. This also advantageously contributes to the patient's well-being and patient safety.

By using methods, systems, and devices of the present disclosure, a fully automated and hygienic method of treating air bubble alarms in the venous line with induced separation of the micro-bubbles can be carried out. This can advantageously help to reduce staff input and increase patient safety.

Air that is inside the disposable and is to be removed can detach itself better from the inner wall of the disposable due to the vibration in which the blood circuit is set. If the contents of the disposable are hereby placed under negative pressure, the volume of the air bubbles increases, which further facilitates their removal. It has also been shown that the applied vibration is particularly beneficial in this regard and promotes the effective detachment of air bubbles and/or air pockets. This contributes significantly to patient safety.

### BRIEF DESCRIPTION OF THE DRAWINGS

The methods, systems, and devices of the present disclosure are described below on the basis of embodiments with reference to the attached drawings. The blood treatment apparatus according to the present disclosure is described using the example of a hemodialysis device, but can also be used in the same way for other blood treatment apparatuses, for example a hemodiafiltration device. In the figures applies:

FIG. 1 shows schematically simplified a fluid line structure of a blood treatment apparatus in a first embodiment;

FIG. 2 shows schematically simplified a part of the blood treatment apparatus of FIG. 1 during the method disclosed herein, which can be carried out by the control device or closed-loop control device of a blood treatment apparatus;

FIG. 3 shows schematically simplified a part of a blood treatment apparatus in a first embodiment;

FIG. 4 shows schematically simplified a part of a blood treatment apparatus in a second embodiment;

FIG. 5 shows schematically simplified a part of a blood treatment apparatus in a third embodiment; and

FIG. 6 shows schematically simplified the course of the method which can be carried out by the control device or closed-loop control device of a blood treatment apparatus.

### DETAILED DESCRIPTION

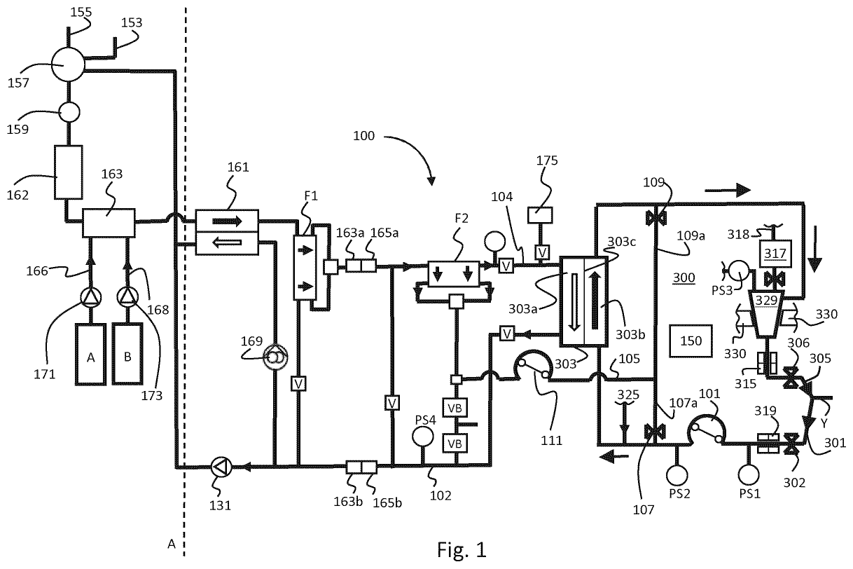

FIG. 1 shows schematically simplified a fluid line structure of a blood treatment apparatus 100 in a first embodiment.

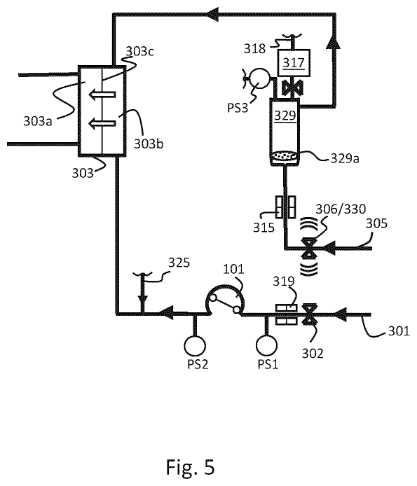

The blood treatment apparatus 100 is connected to an extracorporeal blood circuit 300, which may be connected to the vascular system of the patient, not shown, for treatment by double-needle access (see, for example, FIG. 5), or by single-needle access using, for example, an additional Y-connector (reference numeral Y) as shown, for example, in FIG. 1. The blood circuit 300 may optionally be present in sections thereof in or on a blood cassette.

Pumps, actuators and/or valves in the area of the blood circuit 300 are connected with the blood treatment apparatus 100 or with a control device or closed-loop control device 150, e.g. included by the blood treatment apparatus 100.

The blood circuit 300 includes (or is connected to) an arterial tubing clamp or patient tubing clamp 302 as a first tubing clamp and an arterial connection needle of an arterial section or an arterial patient line, of an arterial line section, of a blood withdrawal line or first line 301. The blood circuit 300 further includes (or is connected to) a venous tubing clamp or patient tubing clamp 306 as a second tubing clamp and a venous connection needle of a venous section, a venous patient line, a venous line section, a blood return line or of a second line 305.

A blood pump 101 is provided in or on the first line 301, a substitute fluid pump 111 for conveying substitute fluid is arranged on a substitute fluid line 105, which may be fluidically connected to a dialysis liquid inlet line 104 for conveying fresh dialysis liquid, which is filtered in a further filter stage F2. Using the substitute fluid pump 111, substitute fluid can be introduced by pre-dilution, via a pre-dilution valve 107, or by post-dilution, via a post-dilution valve 109, via associated lines 107a or 109a into line sections, for example into the arterial line section 301 or into the venous line section 305 (here between a blood chamber 303b of a blood filter 303 and a venous air separation chamber 329 of the blood circuit 300), respectively.

The blood filter 303 includes the blood chamber 303b connected to the arterial line section 301 and to the venous line section 305. A dialysis liquid chamber 303a of the blood filter 303 is connected to the dialysis liquid inlet line 104 leading to the dialysis liquid chamber 303a and to a dialysate outlet line 102 leading away from the dialysis liquid chamber 303a which carries dialysate, e.g., used dialysis liquid. For this purpose, suitable connectors are used on the dialysis liquid inlet line 104 or on the dialysate outlet line 102, respectively, on the one hand and on the dialysate ports on the other hand, which can be connected to one another, e.g., in a detachable manner.

Dialysis liquid chamber 303a and blood chamber 303b are separated from each other by a mostly semi-permeable membrane 303c. It represents the partition between the blood side with the extracorporeal blood circuit 300 and the machine side with the dialysis liquid or dialysate circuit, which is shown in FIG. 1 to the left of the membrane 303c.

The arrangement in FIG. 1 includes an air bubble detector 315 for detecting air and/or blood, e.g., in the venous line section 305, e.g., at the location shown.

The arrangement in FIG. 1 further optionally encompasses one or two pressure sensors PS1, (here upstream of the blood pump 101) and PS2 (downstream of the blood pump 101, it measures the pressure upstream of the blood filter 303 (“pre-hemofilter”)) at the points shown in FIG. 1. Additional pressure sensors can be provided, e.g., the pressure sensor PS3 downstream of the venous air separation chamber 329.

An optional single-needle chamber 317 is used in FIG. 1 as a buffer and/or compensating reservoir in a single-needle procedure in which the patient is connected to the extracorporeal blood circuit 300 using only one of the two blood lines 301, 305.

The arrangement of FIG. 1 also includes an optional detector 319 for detecting air bubbles and/or blood.

An addition site 325 for Heparin may optionally be provided.

On the left in FIG. 1, is shown an optional mixing device 163, which provides a predetermined mixture for the respective solution from the containers A (for A-concentrate via concentrate supply 166) and B (for B-concentrate via concentrate supply 168) for use by the blood treatment apparatus 100. The solution contains water from a water source 155 (on-line, e.g., as reverse osmosis water or from bags) which is heated, for example, in the optional heating device 162.

A pump 171, which can be referred to as a concentrate pump or a sodium pump, can be fluidically connected to the mixing device 163 and a source of sodium, for example the container A and/or convey, if provided, therefrom. An optional pump 173, associated with container B, e.g., for bicarbonate, can be seen.

Furthermore, FIG. 1 shows a waste outlet 153 for the effluent. An optional heat exchanger 157 and a first flow pump 159, which is suitable for de-gassing, complete the arrangement shown.

The optional pressure sensor PS4 downstream of the blood filter 303 on the water side, but, e.g., upstream the ultrafiltration pump 131 as an example of a pump for conveying dialysis liquid and/or dialysate, in the dialysate outlet line 102 may be provided for measuring the filtrate pressure or membrane pressure of the blood filter 303.

Blood that leaves the blood filter 303 flows through a venous blood chamber 329, which may include a de-aeration device 318 and may be in fluid communication with the pressure sensor PS3.

The exemplary arrangement shown in FIG. 1 includes the control device or closed-loop control device 150. The control device or closed-loop control device 150 may be in a wired or wireless signal connection with any of the components mentioned herein—e.g., with the blood pump 101—to control or regulate the blood treatment apparatus 100.

By using the optional device for on-line mixing of the dialysis liquid, a variation of its sodium content, controlled by the control device or closed-loop control device 150, is possible within certain limits. For this purpose, e.g., the measurement values determined by the conductivity sensors 163a, 163b may be taken into account. Should an adjustment of the sodium content of the dialysis liquid (sodium concentration) or of the substitute fluid turn out to be necessary or desired, this can be done by adjusting the conveying rate of the sodium pump 171.

In addition, the blood treatment apparatus 100 includes devices for conveying fresh dialysis liquid and dialysate on the so-called hydraulic side of the blood treatment apparatus 100.

A first valve may be provided between the first flow pump 159 and the blood filter 303, which opens or closes the inlet to the blood filter 303 on the inlet side. A second, optional flow pump 169 is provided, for example, downstream of the blood filter 303, which conveys dialysate to the waste outlet 153. A second valve can be provided between the blood filter 303 and the second flow pump 169, which opens or closes the drain on the outlet side.

Furthermore, the blood treatment apparatus 100 optionally includes a device 161 for balancing the flow flowing into and out of the dialyzer 303 on the machine side. The device 161 for balancing can be arranged in a line section between the first flow pump 159 and the second flow pump 169.

The blood treatment apparatus 100 further includes devices, such as the ultrafiltration pump 131, for the precise removal of a volume of liquid from the balanced circuit, as predetermined by the user and/or by the control device 150.

Sensors such as the optional conductivity sensors 163a, 163b serve to determine the conductivity, which in some embodiments is temperature-compensated, as well as the liquid flow upstream and downstream of the dialyzer 303.

Temperature sensors 165a, 165b may be provided as one or a plurality thereof. Temperature values supplied by them may be used, according to the present disclosure, to determine a temperature-compensated conductivity.

An optional source of compressed air 175, for example in the form of a compressor, may be provided upstream of the blood filter 303 on the machine side.

A leakage sensor 167 is optionally provided. Alternatively, it may also be provided at a different location.

Further flow pumps in addition to or alternatively to e.g. the one with the reference numeral 169 may also be provided.

A number of optional valves are each denoted with V in FIG. 1. Bypass valves with VB.

Based on the measurement values of the above-mentioned, optional sensors, the control device 150 determines in some embodiments the electrolyte and/or fluid balance.

Filters F1 and F2 can be provided connected in series.

Even when using non-pure water, the filter F1 exemplarily serves here to generate sufficiently pure dialysis liquid by the mixing device 163, which then flows through the blood filter 303, e.g. using the countercurrent principle.

The filter F2 exemplarily serves here to generate sterile or sufficiently filtered substitute fluid from the sufficiently pure dialysis liquid leaving the first filter F1, by filtering, e.g., pyrogenic substances. This substitute fluid may then be safely added to the extracorporeally flowing blood of the patient and thus ultimately to the patient's body.

The blood treatment apparatus 100 which is shown in FIG. 1 can be a hemofiltration apparatus, a hemodiafiltration apparatus or a hemodialysis apparatus.

The present disclosure is not limited to the embodiment described above, which is for illustrative purposes only.

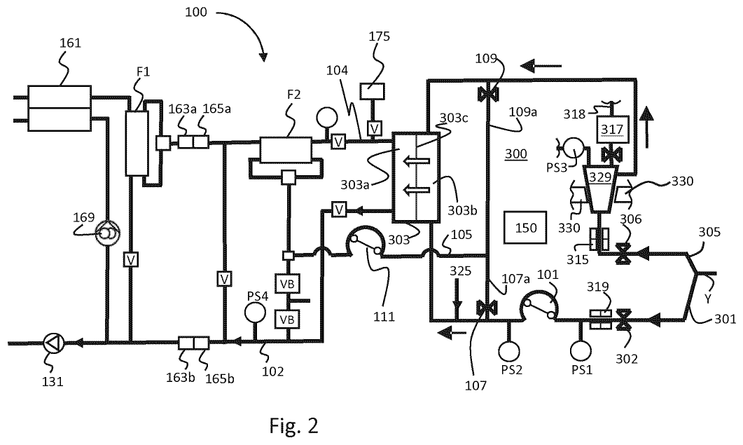

The arrows and arrowheads shown in FIG. 1 generally indicate the direction of flow in FIG. 1 and FIG. 2.

The arterial line section 301 and the venous line section 305 may be part of or form a set of blood lines.

FIG. 2 shows schematically simplified a part of the blood treatment apparatus 100 of FIG. 1, namely the part to the right of the dashed line A, when carrying out the method disclosed herein.

To avoid repetition, reference is made to the description of FIG. 1.

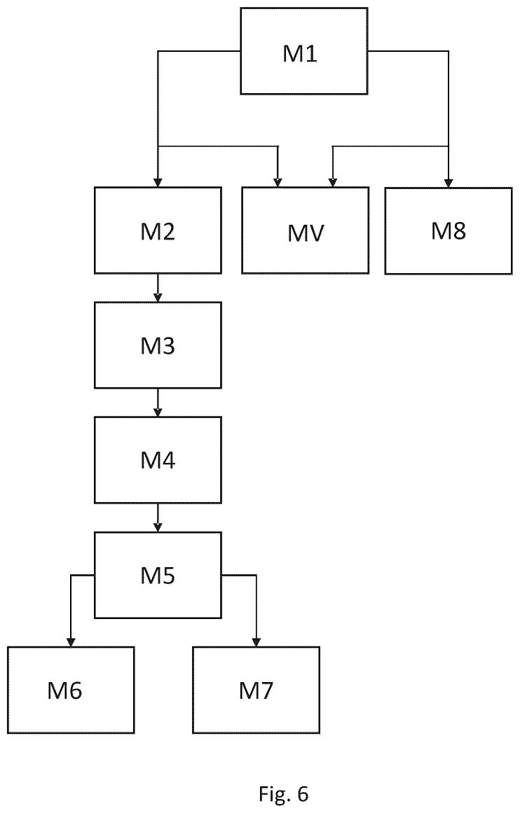

The method, which is described in more detail in FIG. 6, results in flow reversal in the venous line section 305, as indicated by the reversed direction of the arrows and arrowheads compared to FIG. 1, to prevent or significantly reduce the flow between the venous tubing clamp 306 and the patient, and in the transfer of fluid from the blood chamber 303b to the dialysis liquid chamber 303a, as also indicated by arrows.

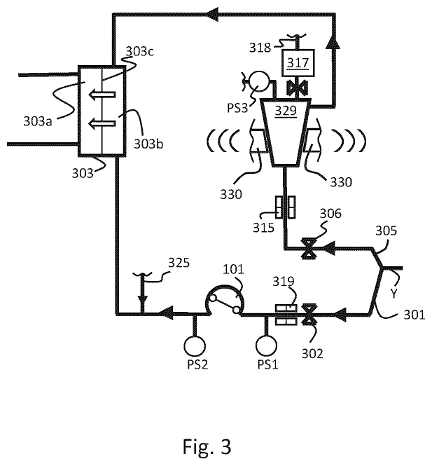

FIG. 3 shows schematically simplified a part of a blood treatment apparatus 100, e.g., the extracorporeal blood circuit 300, in the embodiment of FIGS. 1 and 2.

A chamber holder can be seen to the left and right of the venous air separation chamber 329, which can hold the venous air separation chamber 329 securely in its position during use, for example on a blood cassette. In this embodiment, this chamber holder serves as an example of an vibration device 330 in that it itself is displaced, oscillated or moved, for example via optionally integrated actuators, e.g., it includes a stroke. These actuators can be generated, for example, via a vibration motor, sound transducers (e.g., generated by piezoceramics or similar sound-generating materials) and/or electroactive polymers. The chamber holder thus set in vibration transmits this vibration to the venous air separation chamber 329, or its contents.

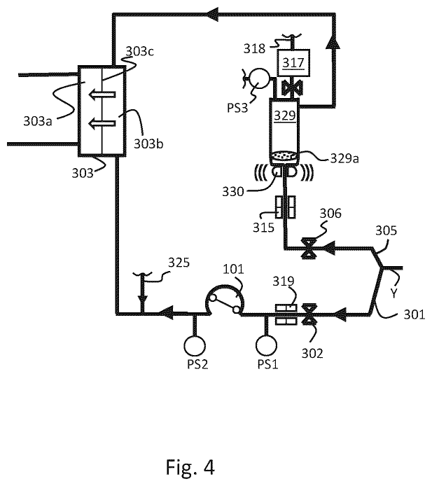

FIG. 4 shows schematically simplified a part of a blood treatment apparatus 100, e.g., the extracorporeal blood circuit 300, in a second embodiment.

FIG. 4 corresponds in large parts to FIG. 3, so only the differences will be discussed here in order to avoid repetition.

The venous air separation chamber 329 is more cylindrical in shape. A sieve-shaped clot trap 329a is shown at its outlet (bottom of FIG. 4). Air that has been conveyed across the clot trap 329a downstream towards the patient can only rise again across this with difficulty.

If the venous air separation chamber 329, its clot trap 329a or the liquid contained therein is set in motion, e.g. vibration, the air can be moved more easily across the sieve (mesh) of the clot trap 329a back into the venous air separation chamber 329 and may be separated there via the de-aeration device 318, for example into the atmosphere.

In this embodiment, the venous air separation chamber 329 is held by a bracket, for example in the form of a fork, which can hold the venous air separation chamber 329 securely in position during use, for example on a blood cassette. In this embodiment, this fork-shaped holder corresponds to an example of a vibration device 330. It can easily be set in motion/vibration itself, for example by or analogously to the actuators already explained with regard to FIG. 3. The “fork” set in motion in this way transmits the vibration to the venous air separation chamber 329, e.g., to the clot trap 329a, or its contents.

FIG. 5 shows schematically simplified a part of a blood treatment apparatus 100, e.g., the extracorporeal blood circuit 300, in a third embodiment.

FIG. 5 largely corresponds to FIG. 3 or FIG. 4, so only the differences will be discussed here in order to avoid repetition.

In contrast to FIGS. 3 and 4, in the example in FIG. 5 the extracorporeal blood circuit 300 is connected to the vascular system of the patient (not shown) for treatment by means of double-needle access.

Further, in this embodiment, the vibration device 330 is or includes the venous tubing clamp 306. In this embodiment, the control device or closed-loop control device 150 is programmed to control the venous tubing clamp 306 so that the venous tubing clamp 306 is automatically opened and closed or is otherwise actuated multiple times within a minute or a second.

This opening and closing may generate sufficient vibration, oscillation, or motion that detected air bubbles or air pockets may be released or such a process may be assisted.

FIG. 6 shows schematically simplified a course of the method which can be carried out by the control device or closed-loop control device 150 of a blood treatment apparatus 100.

In the description of the method, reference is made to the reference numerals from FIGS. 1 to 4.

The blood treatment apparatus 100, which is to be controlled by using the method, has a blood pump 101, an air bubble detector 315 and a pump for conveying dialysis liquid and/or dialysate on the hydraulic side of the blood treatment apparatus 100 (see FIGS. 1 and 2). The method is intended to be carried out during an extracorporeal blood treatment session, e.g., intradialytically. Meanwhile, the blood treatment apparatus 100 is connected to an extracorporeal blood circuit 300 and to a blood filter 303. The blood filter 303 in turn includes a semi-permeable membrane 303c.

Hereby, the method includes a detection of air bubbles or air pockets in the extracorporeal blood circuit 300, or an air bubble alarm (M1).

Air bubbles or air pockets in the extracorporeal blood circuit 300 can be detected by the air bubble detector 315. Alternatively or additionally, the air bubble detector 315 can be suitable or configured to trigger an air bubble alarm (alternatively: gas alarm) as a result of detecting gas, air or air bubbles, which in turn causes the method to be initiated or carried out.

If a gas alarm occurs, the blood treatment may be stopped or suspended until the air has been removed from the extracorporeal blood circuit 300.

If air bubbles or air pockets have been detected in the extracorporeal blood circuit 300 or if an air bubble alarm is present, the control device or closed-loop control device 150 causes the blood pump 101 to stop or to reduce its conveying rate with which it conveyed just before the air bubbles have been detected (M2).

Optionally, the method can include a complete or partial closing of the venous tubing clamp 306 (M3).

Furthermore, a negative transmembrane pressure is generated across the semi-permeable membrane 303c of the blood filter (M4). This negative transmembrane pressure can be achieved by appropriately controlling the pump, here for example the ultrafiltration pump 131, to convey dialysis liquid and/or dialysate on the hydraulic side, while the blood pump 101 is stopped or its conveying rate is reduced.

M5 represents an opening of the venous tubing clamp 306 when negative transmembrane pressure has already been generated while maintaining a negative transmembrane pressure or when negative transmembrane pressure is present.

In several embodiments, the venous tubing clamp 306 is opened if a minimum value of a negative transmembrane pressure has been detected, for example by at least one pressure sensor PS4 of the blood treatment apparatus 100.

In some embodiments, the pressure sensor PS3 (venous sensor) or the pressure sensor PS2 will be used to assess the pressure situation. In these as well as in any other embodiments, the detected pressures may be monitored to detect a pressure drop and to open the venous tubing clamp 306 accordingly. Experience has shown that the operating pressure during treatment is mostly around 320 mmHg for PS3, and around 300 mmHg for PS2.

Due to the negative transmembrane pressure, a (transmembrane) plasma water transfer takes place via the membrane 303c and the blood hemoconcentrates in the dialyzer 303. Hereby, the volume pumped into the hydraulic system is replaced by the blood flowing out of the patient via the venous tubing system in the opposite direction to the usual flow. The air bubbles or air pockets (gas bubbles and micro-bubbles) present in the line are conveyed back into the venous air separation chamber 329 and separated there, for example into the environment.

In certain embodiments, negative transmembrane pressure is generated by the pump while an optional substitute fluid pump 111 of the blood treatment apparatus 100 is stopped or is not conveying.

M6 represents stopping the generation or maintenance of the negative transmembrane pressure once a predetermined negative transmembrane pressure, which is for example in the range between −300 mmHg and −500 mmHg, has been reached or a volume of at least 30 to 60 ml of blood has been conveyed towards an arterial needle or a venous air separation chamber 329 by, or due to, the generated transmembrane pressure.

Alternatively, in M7, conveying by the pump to generate a negative transmembrane pressure is terminated after the air bubble detector 315 no longer detects any air bubbles or air pockets and/or there is no longer an air bubble alarm.

Simultaneously or overlapping with one, more or all of the method parts M2 to M7, the control device or closed-loop control device 150 of the blood treatment apparatus 100 may cause the vibration device 330 to vibrate, oscillate or move in a similar way the extracorporeal blood circuit 300 or at least a portion thereof, or respectively the contents or part of the contents of the blood circuit or portion. This is illustrated in FIG. 6 by MV. Also encompassed is causing the above vibrations without performing the method parts M2 to M7.

Alternatively, in certain embodiments, when air bubbles or air pockets are detected or when an air bubble alarm is present, the control device or closed-loop control device 150 causes the conveying direction of the blood pump to be reversed, e.g., the blood pump conveys backwards. This is shown in FIG. 6 as M8. In this embodiment—as optionally in any other embodiment—reverse conveying could alternatively or additionally take place by generating a negative transmembrane pressure, for example by suitable control of balance chamber circuits, whereby the balance chambers are usually designed as diaphragm pumps.

Simultaneously, alternatively or overlapping with this alternative, e.g., M8, the control device or closed-loop control device 150 may also initiate MV and cause the extracorporeal blood circuit 300 or portions thereof to vibrate, move or oscillate as described above.

### LIST OF REFERENCE NUMERALS

100 blood treatment apparatus
101 blood pump
102 dialysate outlet line
104 dialysis liquid inlet line
105 substitute fluid line
107 pre-dilution valve
107a line belonging to the pre-dilution valve
109 post-dilution valve
109a line belonging to the post-dilution valve
111 substitute fluid pump
131 ultrafiltration pump
150 control device
153 waste outlet
155 water source
157 heat exchanger
159 first flow pump
161 balancing device
162 heating device
163 mixing device
163a conductivity sensor
163b conductivity sensor
165a temperature sensor
165b temperature sensor
166 concentrate supply
167 leakage sensor
168 concentrate supply
169 second flow pump
171 pump; sodium pump
173 pump; bicarbonate pump
175 compressed air source; compressor
300 extracorporeal blood circuit
301 first line (arterial line section)
302 (first) tubing clamp; arterial tubing clamp
303 blood filter or dialyzer
303a dialysis liquid chamber
303b blood chamber
303c semi-permeable membrane
305 second line (venous line section)
306 (second) tubing clamp; venous tubing clamp
315 air bubble detector
317 single-needle-chamber
318 de-aeration device
319 detector
325 addition point for Heparin
329 venous air separation chamber
329a blood clot trap
330 vibration device
F1 filter
F2 filter
A holder
B holder
PS1 arterial pressure sensor (optional)
PS2 arterial pressure sensor (optional)
PS3 pressure sensor (optional)
PS4 pressure sensor for measuring the filtrate pressure (optional)
M1 to M8 method steps
MV method step
V valves
VB bypass valves
Y Y-connector

1-17. (canceled)

18. A blood treatment apparatus comprising:
a blood pump for pumping blood through an extracorporeal blood circuit during a blood treatment session, during which the blood treatment apparatus is connected to the extracorporeal blood circuit and to a blood filter comprising a semi-permeable membrane;
an air bubble detector for detecting air bubbles inside the extracorporeal blood circuit;
a pump for conveying dialysis liquid and/or dialysate;
a vibration device for causing the extracorporeal blood circuit, a section thereof, or contents thereof to vibrate; and
a control device or closed-loop control device for controlling the blood pump, the pump for conveying dialysis liquid and/or dialysate, and the vibration device.

19. The blood treatment apparatus according to claim 18, wherein the vibration device is arranged in or on the extracorporeal blood circuit or arranged for acting upon the extracorporeal blood circuit to cause the extracorporeal blood circuit, the section thereof, or the contents thereof to vibrate.

20. The blood treatment apparatus according to claim 18, wherein:
the vibration device is or encompasses a venous tubing clamp; and
the control device or closed-loop control device is programmed to control the venous tubing clamp to automatically open and close several times within a minute or within a second.

21. The blood treatment apparatus according to claim 18, wherein the vibration device is or comprises a holding device for releasably holding at least a section of the extracorporeal blood circuit.

22. The blood treatment apparatus according to claim 18, wherein the control device or closed-loop control device is configured to modulate, or to prompt the modulation of, frequency of the vibrations generated by the vibration device.

23. The blood treatment apparatus according to claim 18, wherein the blood treatment apparatus is connected to the extracorporeal blood circuit and a blood filter having the semi-permeable membrane.

24. The blood treatment apparatus according to claim 23, wherein the extracorporeal blood circuit is or comprises a blood tubing set and/or a blood cassette.

25. The blood treatment apparatus according to claim 23, wherein the blood treatment apparatus is a hemodialysis apparatus, hemofiltration apparatus, hemodiafiltration apparatus, or as an apparatus for carrying out a separation procedure.

26. A method configured to be carried out on a blood treatment apparatus, the method comprising:
detecting air bubbles in an extracorporeal blood circuit connected to the blood treatment apparatus; and
in response:
stopping a blood pump of the blood treatment apparatus or reducing a conveying rate of the blood pump;
generating a negative transmembrane pressure across a semi-permeable membrane of the blood treatment apparatus by appropriately controlling a pump for conveying dialysis liquid and/or dialysate, while a blood pump of the blood treatment apparatus is stopped or the conveying rate of the blood pump is reduced; or
reversing a conveying direction of the blood pump,
wherein:
the blood pump of the blood treatment apparatus is configured for pumping blood through the extracorporeal blood circuit during a blood treatment session, during which the blood treatment apparatus is connected to the extracorporeal blood circuit and to a blood filter comprising a semi-permeable membrane; and
the blood treatment apparatus comprises:
an air bubble detector for detecting air bubbles inside the extracorporeal blood circuit;
the pump for conveying dialysis liquid and/or dialysate;
a vibration device for causing the extracorporeal blood circuit, a section thereof, or contents thereof to vibrate; and
a control device or closed-loop control device for controlling the blood pump, the pump for conveying dialysis liquid and/or dialysate, and the vibration device.

27. The method according to claim 26, wherein the negative transmembrane pressure is generated across the semi-permeable membrane when a venous tubing clamp of the blood treatment apparatus is completely or partially closed.

28. The method according to claim 26, further comprising opening a venous tubing clamp of the blood treatment apparatus when the negative transmembrane pressure has already been generated while maintaining a negative transmembrane pressure.

29. The method according to claim 28, wherein the venous tubing clamp is opened if a pressure sensor of the blood treatment apparatus has determined that a minimum value of a negative transmembrane pressure has been reached.

30. The method according to claim 26, wherein the negative transmembrane pressure is reached by using the pump for conveying dialysis liquid and/or dialysate while a substitute fluid pump of the blood treatment apparatus is stopped or is not conveying.

31. The method according to claim 26, wherein generating the negative transmembrane pressure is terminated as soon as a predetermined negative transmembrane pressure is reached or a volume of at least 30 to 60 ml or more blood has been conveyed in a direction of an arterial connection needle or a venous air separation chamber by or due to the transmembrane pressure.

32. The method according to claim 31, wherein the predetermined negative transmembrane pressure is between −300 mmHg and −500 mmHg.

33. The method according to claim 26, wherein generating a negative transmembrane pressure is terminated after the air bubble detector no longer detects air bubbles or air pockets, and/or no air bubble alarm is present.

34. The method according to claim 26, wherein at least one of detecting the air bubbles, stopping the blood pump, generating the negative transmembrane pressure, and reversing the conveying direction is overlapping in time.

35. The method according to claim 26, wherein the pump for conveying dialysis liquid and/or dialysate is an ultrafiltration pump.
